(19) United States
(12) Patent Application Publication
(10) Pub. No.: US 2021/0015496 A1
(43) Pub. Date: Jan. 21, 2021

(54) DRILL BIT FOR DENTAL IMPLANT SURGERY

(71) Applicant: Taekyoung LEE, Seoul (KR)

(72) Inventor: Taekyoung LEE, Seoul (KR)

(21) Appl. No.: 17/062,590

(22) Filed: Oct. 4, 2020

Related U.S. Application Data
(63) Continuation-in-part of application No. PCT/KR2019/003981, filed on Apr. 4, 2019.

(30) Foreign Application Priority Data
Apr. 4, 2018 (KR) 10-2018-0038979

(51) Int. Cl. A61B 17/16; A61C 8/00

(52) U.S. Cl. CPC A61B 17/1673; A61B 2090/033; A61C 8/0089

(57) ABSTRACT

Provided is a drill bit for implant surgery including: a cutting part including first and second cutting elements, wherein each of the first and the second cutting elements has a predetermined thickness, wherein a flute is formed between the first and the second cutting elements; a guide part extending from the cutting part, being in a cylindrical shape, and having a constant diameter; a peripheral part extending from the guide part, having a predetermined length, and having the same diameter as that of the guide part; and a shank part extending from the peripheral part and having a different diameter from that of the peripheral part to form a step between the peripheral part and the shank part.

### CROSS REFERENCE TO RELATED APPLICATIONS

This application is a continuation in part application of International Application No. PCT/KR2019/003981, filed on Apr. 4, 2019, which claims priority to Korean Application No. 10-2018-0038979, filed on Apr. 4, 2018, the entire contents of which are herein incorporated by reference in its entirety.

### TECHNICAL FIELD

The present invention relates to a drill bit for dental implant surgery and, more specifically, to a drill bit for dental implant surgery that is particularly suitable for precision guided implant surgery.

### BACKGROUND OF INVENTION

Dental implants (hereinafter, simply referred to as “implant”) are also referred to as artificial teeth or third teeth.

In other words, an implant refers to an artificial tooth itself or a dental treatment technique. The dental treatment technique restores the function of natural teeth by implanting artificial teeth made of a material with excellent biocompatibility, for example, a titanium-based metal material in a jawbone where teeth were extracted or where tooth cavities are located.

If the jawbone at the site where the implant is to be placed is insufficient, the volume of the bone tissue is increased to sufficiently wrap the implant by additional surgery such as bone transplant and bone expansion surgery.

These implants have various structures. According to published literature, generally, they are composed of a fixture, an abutment, and an artificial crown.

The fixture is in a screw shape and is made of a material with excellent biocompatibility. The fixture is implanted into the cavity in an alveolar bone where the tooth was lost, and is fused with the bone. The abutment is an upper structure and coupled to the fixture by a screw coupling method or by a force-fitting method. An artificial crown is mounted on the abutment for mastication and cosmetic benefits.

In this way, the implant is structurally and functionally composed of approximately three parts. Two factors are important for a successful implant procedure: first, how firmly and compactly the fixture is implanted into the alveolar bone as planned; second, how securely the fixture surface is fused with the bone so as to perform as cohesively as natural teeth.

In other words, the implant can only function as a long-term artificial tooth if the fixture that forms the basis of the implant and receives most of the load during mastication is implanted firmly enough in the alveolar bone.

Incomplete fixation of a fixture occurs mainly due to an excessive space between the fixture and the alveolar bone. Excessive space leads an inflammatory tissue or soft tissue to grow, more rapidly than the alveolar bone cells. Under this condition, the soft tissue or the inflammatory tissue or microbes may adhere to the fixture surface and grow rapidly before bone fusion is achieved. Eventually, the implant may fall out or must be removed unavoidable, and it even makes reoperation impossible.

Considering that the high cost of an implant procedure, the importance of firmly securing the fixture into the alveolar bone cannot be overemphasized.

In addition, the fixture should be safely positioned so that anatomical tissues adjacent to the alveolar bone, such as neural tubes or maxillary sinuses, are not damaged. In addition, the fixture must be positioned so that the thickness of the surrounding alveolar bone is sufficient.

When the thickness of the surrounding alveolar bone is not sufficient, artificial bone graft surgery is performed. The artificial bone should be biomechanically appropriate located in relation to the alveolar bone and fixture. To meet this requirement, precision guided surgery has been adopted and is used more and more.

The implant surgery drill bit used in precision guided surgery has a blade corresponding to the shape of the fixture. If the fixture is tapered, the corresponding blade of the implant surgery drill bit also has a tapered shape. Due to such limitation, to guide the drill bit in a cutting direction has to be made by a separate guiding part. This lowers guiding precision during the cutting process.

For this reason, in the case of a tapered drill bit, there is a high possibility that the fixture will be placed in an unsafe or physiologically unfavorable position. This may lead to an implant operation failure.

For this reason, it is recommended to use a straight type drill bit which is a good guiding characteristic. A conventional twist drill bit has the spiral flute. The spiral flute degrades the guiding functionality. A drill bit that does not have a flute on its body is advantageous in improving guiding functionality in implant precision guided surgery.

Therefore, there is a need to change a conventional implant surgical drill bit to a flute-free straight drill bit.

In addition, in designing an implant surgery drill bit for implant surgery, several factors must be considered, including: the anatomical characteristics of the alveolar bone; the method of implant procedure in which the perforation of the alveolar bone is completed over multiple steps; safety measures to minimize the risk to the patient during use; and the characteristics of precision guided implant surgery in which guide bushings are used. However, it is difficult to find a drill bit for implant surgery that comprehensively satisfies such requirements.

### DETAILED DESCRIPTION OF INVENTION

### Technical Problems

An objective of the present invention is to provide a drill bit for dental implant surgery that reinforces the guiding function during precise guided implant surgery. The drill bit is designed taking into consideration: the anatomical characteristics of the alveolar bone; the method of implant procedure in which the perforation of the alveolar bone is completed over multiple steps; safety measures to minimize the risk to the patient during use; and the characteristics of precision guided implant surgery in which guide bushings are used.

### SUMMARY OF INVENTION

The present invention provides a drill bit for implant surgery comprising: a cutting part including first and second cutting elements, wherein each of the first and the second cutting elements has a predetermined thickness, wherein a flute is formed between the first and the second cutting elements; a guide part extending from the cutting part, being in a cylindrical shape, and having a constant diameter; a peripheral part extending from the guide part, having a predetermined length, and having the same diameter as that of the guide part; and a shank part extending from the peripheral part and having a different diameter from that of the peripheral part to form a step between the peripheral part and the shank part.

The first cutting element includes a first flat tip, a first inclined portion, and a first corner. The second cutting element includes a second flat tip, a second inclined portion, and a second corner. A vertical direction is defined as a longitudinal direction of the drill bit. A horizontal direction is perpendicular to the vertical direction. Each of the first and the second flat tips extends in a horizontal direction. The first and the second flat tips overlap each other.

The first and the second inclined portions obliquely extend outward from the first and the second flat tips, respectively. The first and the second corners extend from the first and the second inclined portions, respectively, along the vertical direction. Each of the first and the second corners has the same diameter as that of the guide part. No cutting edge is provided on either of the first and the second corners.

At least one of the first and the second elements includes a flat tip cutting edge and an inclined portion cutting edge. The flat tip cutting edge is provided on the first flat tip or on the second flat tip. The inclined portion cutting edge is provided on the first inclined portion or on the second inclined portion.

A first flat tip cutting edge and a first inclined portion cutting edge are provided on the first flat tip and the first inclined portion of the first cutting element, respectively. The first flat tip cutting edge protrudes the farthest from a front end of the drill bit.

The length of the second flat tip of the second cutting element is substantially the same as the thickness of the first flat tip of the first cutting element. The second flat tip of the second cutting element is buried under the first flat tip cutting edge.

A second inclined portion cutting edge is provided on the second inclined portion of the second cutting element.

The first flat tip and the second flat tip overlap each other. A first inclined portion cutting edge is provided on the first inclined portion of the first cutting element. A second inclined portion cutting edge is provided on the second inclined portion of the second cutting element.

The drill bit for implant surgery may further comprise a first flat tip cutting edge which is formed on the first flat tip of the first cutting element. The first flat tip cutting edge is divided into two by the second flat tip of the second cutting element.

An end of the first flat tip cutting edge of the first cutting element extends up to or shorter than a point where the second flat tip of the second cutting element is located.

A functional groove is concavely formed on an outer surface of the guide part. The functional groove is connected to the flute. The functional groove is (i) in a straight line shape extending along the longitudinal direction of the drill bit, or (ii) in a spiral shape extending around the guide part in a clockwise or counterclockwise direction. The functional groove in the spiral shape is formed shorter than one rotation around the guide part.

The drill bit for implant surgery may further comprise an engraving formed in an intaglio on an outer surface of the guide part. The engraving includes one or more symbols. The number of symbols indicates a length of the drill bit. The engraving is formed by a cutting process in which the cutting part is formed.

The drill bit for implant surgery may further comprise an identification groove formed in an intaglio on a circumferential surface of the peripheral part. The identification groove is spaced apart by a predetermined distance from the step. The identification groove indicates a diameter of the drill bit. The identification groove is formed by a cutting process in which the cutting part is formed. The identification groove includes multiple grooves. The multiple grooves are spaced apart from each other by a predetermined interval.

The drill bit for implant surgery may further comprise a stopper protruding outwardly from the step.

Each of the first flat tip and the second flat tip is a flat surface. A water hole is provided penetrating from the end of the shank part to the flat surface.

No flute is provided on the guide part. The flute does not extend onto the guide part.

### Advantages of Invention

An implant surgery drill bit of the present invention having the configuration as described above does not have to form a flute in the body of the cylindrical drill bit. Instead, one or more flutes are provided on cutting elements, considering the anatomical characteristics of the alveolar bone. As a result, implant precision guidance improves in a surgery using a guidance bushing, thereby ensuring precise implant surgery according to a given procedure plan.

In addition, since the lateral cutting force can be suppressed, unnecessary abrasion between an inner surface of the guide bushing and the implant surgery drill bit can be prevented. In addition, expansion of the alveolar bone caused by the tolerance between the inner surface of the guide bushing and the drill guide can be suppressed.

In addition, due to an improved structural shape of the cutting elements, there is almost no fear that the implant surgery drill bit will invade the neural tube or the side wall of the maxillary sinus, thereby improving the safety of the patient surgery.

In addition, the functional groove formed symmetrically reduces the frictional force between the inner surface of the guide bushing and the drill guide part, while minimizing movement and vibration due to tolerances, and securing a water supply path through the functional groove.

In addition, in the implant surgery drill bit for implant surgery of the present invention, the cutting elements have a structure suitable for an alveolar bone drilling operation which is performed over multiple steps. Various types of drill bits can be employed and they are distinguished from each other by inscriptions and grooves formed on each drill bit. The inscriptions and grooves indicate length and diameter of a given implant surgery drill bit, respectively. Due to the inscriptions and grooves, a proper drill bit necessary for each step can be selected accurately and quickly in the process of the surgery. As a result, the efficiency of surgery can be improved. Compared with a conventional art which marks the inscriptions and grooves which are formed in a separate post process, manufacturing efficiency is enhanced and the risk of erroneous marking can be significantly reduced in the present invention.

### BRIEF DESCRIPTION OF THE DRAWING

FIG. 1 shows a drill bit for implant surgery according to a first embodiment of the present invention.

FIG. 2 shows a drill bit for implant surgery according to a second embodiment of the present invention. FIG. 2 shows different cutting elements from FIG. 1.

FIGS. 3A-3C shows various functional grooves which are formed on a guide part of a drill bit for implant surgery.

FIG. 4 shows a drill bit coupled with a guide bushing.

FIG. 5 shows a drill bit for implant surgery according to a third embodiment to the present invention.

FIG. 6 shows a drill bit for implant surgery according to a fourth embodiment to the present invention. The drill bit for implant surgery shown in FIG. 6 is different from the drill bit for implant surgery shown in FIG. 5 in the structure of the cutting elements.

FIG. 7 shows a drill bit for implant surgery according to a fifth embodiment to the present invention. The drill bit for implant surgery shown in FIG. 7 is provided with a water hole.

Embodiments of the present invention will be described in detail in reference to the accompanying drawings.

In adding reference numerals to elements of each drawing, it should be noted that the same elements are assigned with the same numerals as possible even if they are indicated on different drawings.

### EXPLANATION OF REFERENCE NUMERALS

10: implant surgery drill bit
12: front end
100: cutting part
110: cutting element
111: first cutting element
112: second cutting element
114: flat tip
115: inclined part
116: corner
118: cutting edge
119: flute
200: guide part
210: functional groove
212: water hole
220: engraving
300: peripheral part
310: identification groove
400: shank part
410: stopper
GB: guide bushing

### EMBODIMENTS

Hereinafter, in describing embodiments of the present invention, when it is determined that a detailed description of a known configuration or function may hinder an understanding of the embodiments of the present invention, the detailed description thereof will be omitted.

In addition, in describing the elements of the embodiments of the present invention, terms such as first, second, A, B, (a), and (b) may be used.

These terms are only used to distinguish the elements from each other. The nature or order of the elements is not limited by the term.

When an element is described as being “connected” or “coupled” to another element, the element may either be directly connected to that other element, or indirectly connected, with a third element between the two elements.

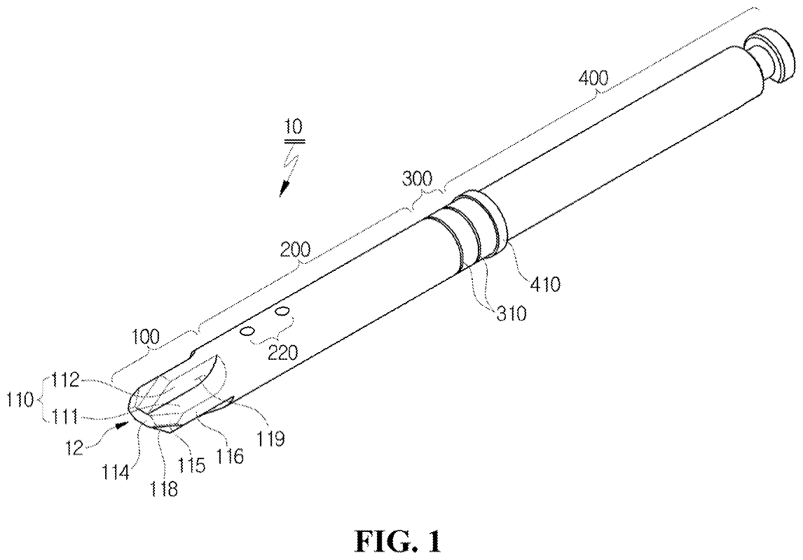

FIG. 1 shows an implant surgical drill bit (10) according to a first embodiment of the present invention. With reference to FIG. 1, the present invention will be described in detail below.

For reference, the description of the second embodiment of the present invention will be focused on a configuration distinct from the first embodiment. In addition, since there are many basic structural similarities between the implant surgical drill bit (10) illustrated in the first and second embodiments, it should be understood that the description of the first embodiment can also be applied to the second embodiment unless otherwise specified.

Referring to FIG. 1, the implant surgery drill bit (10) of the present invention includes a cutting part (100), a guide part (200), a peripheral part (300), and a shank part (400).

Here, the major difference between the first embodiment and the second embodiment is found in the cutting part (100).

FIG. 1 shows a first embodiment of an implant surgery drill bit (10) for implant surgery. The cutting part (100) is located at a front end (12) of the implant surgery drill bit (10). The cutting part (100) includes two or more cutting elements (110).

Each of the cutting elements (110) includes a flat tip (114), an inclined portion (115), and a corner (116). A vertical direction is defined as a longitudinal direction of a given drill bit.

A horizontal direction is perpendicular to the vertical direction.

In each of the cutting elements: the flat tip (114) extends in a horizontal direction; the inclined portion (15) obliquely extends outward from the flat tip; and the corner (116) extends from the inclined portion along the vertical direction,

The cutting element (110) has a predetermined thickness. Accordingly, the individual cutting element (110) as a whole is in a plate-like shape with a predetermined thickness.

Here, the “cutting elements (110)” or “cutting element (110)” should be understood as a component provided in the cutting part (100). A cutting edge (118), which causes a cutting action in the implant surgery drill bit (10), can be formed on the “cutting element (110).

The cutting edge (118) may be formed only on some of multiple cutting elements (110). That is, not all cutting elements (110) have the cutting edge (118).

The cutting elements (110) are arranged to cross each other so that the flat tips (114) of the cutting elements overlap each other. The cutting edges (118) are formed on the flat tip (114) and on the inclined portion (115) of one or more of the cutting elements (110).

An important feature of the implant surgery drill bit (10) of the present invention is that the flat tip (114) is a flat surface.

This is a significant difference from a conventional drill bit. In a conventional drill bit, a cutting part is in a conical shape.

As described above, the present invention introduces the flat tip (114) to the cutting part (100), making the front end flat. Under this structure, the front end (12) of the implant surgery drill bit (10) does not invade the neural tube or the side wall of the maxillary sinus when drilling the alveolar bone.

Another reason why the flat tip (114) is formed of a flat surface is because of the anatomical features of the alveolar bone.

The surface of the alveolar bone is a dense and hard cortical bone. However, its thickness is only 1 to 4 mm. Most of the alveolar bone is made up of relatively less dense and weak cancellous bone.

Therefore, even when the front end (12) of the implant surgery drill bit (10) is flat, the overall drilling operation can be performed smoothly once the thin bone cortex is drilled. Thus, in many aspects, it is advantageous to make the front end (12) flat by employing the flat tip (114) to the implant surgery drill bit (10).

Further, a flute (119) is formed between neighboring cutting elements (110) and serves as a space temporarily accommodating bone fragments which are generated when the alveolar bone is drilled, thereby preventing the bone fragments from interfering with the cutting process.

Since most of the alveolar bone is relatively less dense cancellous bone, only the flute (119) space provided between the cutting elements (110) is sufficient to accommodate the bone fragment during the perforation operation. In light of this fact, in the present invention, it is not necessary to form a spiral flute (119) on the guide part (200) for discharging bone fragments. In contrast, the conventional twist drill bit has a long spiral flute which is formed along the body.

The guide part (200) without flute ensures the precision of the guided surgery method that uses a guide bushing (GB). Such an advantage will now be discussed in detail.

Each of the part of the cutting elements (110) extends along a vertical direction from the inclined part (115). The corner (116) is also part of the cutting element (110). According to the present invention, no cutting edge (118) is formed on the corner (116).

An outer surface of the corner (116) is smoothly connected to the guide part (200). In the initial stage of the cutting process where the guide part has not yet reached the inner peripheral surface of the guide bushing, the corner (116) contacts the inner circumferential surface of the guide bushing (GB) and guides the implant surgery drill bit (10) to the alveolar bone accurately.

In addition, it is preferable that the cutting edges (118) formed on the inclined portions (115) of the cutting elements (110) be disposed on the circumference of the implant surgery drill bit (10) at an isometric angle as being spaced apart from each other by an equal distance.

The cutting elements (110) are intersected at a front end of the drill bit. The inclined portions (115) are provided along the circumference of the implant surgery drill bit (10). Under this structure, it is preferable that the inclined portion cutting edges (118) of the inclined portions (115) are disposed at an isometric angle. This structure equally distributes the adverse impact either in period or in size which is generated during the cutting process. In addition, the structure is advantageous in minimizing the adverse effect on work precision.

Here, the fact that the cutting edges (118) of the inclined portions (115) are arranged at an isometric angle does not necessarily mean that the multiple cutting elements (110) must be arranged at an isometric angle.

Even when the inclined portions (115) are not arranged at an isometric angle, the inclined portion cutting edges (118) still can be arranged at an isometric angle. For example, the inclined portion cutting edges (118) can be provided on some of the inclined portions (115) in an alternating manner.

Of course, it is most preferable that the inclined portion cutting elements (110) are also arranged at an isometric angle.

The drawings attached to the present application show an embodiment in which the cutting elements (110) include a first cutting element (111) and a second cutting element (112). However, the present invention is not limited thereto. A third and/or a fourth cutting element and more may also be added in a similar manner.

In reference to the embodiment shown in FIG. 1, a first flat tip cutting edge (118) is formed on a first flat tip (114) and an inclined portion cutting edge (118) is formed on an inclined portion (115) of the first cutting element (111). No cutting edge (118) is formed on the second cutting element (112). The first flat tip cutting edge (118) is formed on the first flat tip (114) of the first cutting element (111). The first flat tip cutting edge (118) protrudes the farthest from the front end (12), thereby forming an apex. That is, the first cutting element (111) predominantly contributes the cutting action.

The length of the second flat tip (114) of the second cutting element (112) corresponds to the width of the first flat tip (114) of the first cutting element (111). Accordingly, the second flat tip (114) of the second cutting element (112) is hidden under the first flat tip (114) of the first cutting element (11).

That is, in the first embodiment shown in FIG. 1, the second flat tip (114) of the second cutting element (112) appears as if not existing. Under this structure, the main function of the second cutting element (112) is to reinforce structural strength of the first cutting element (111).

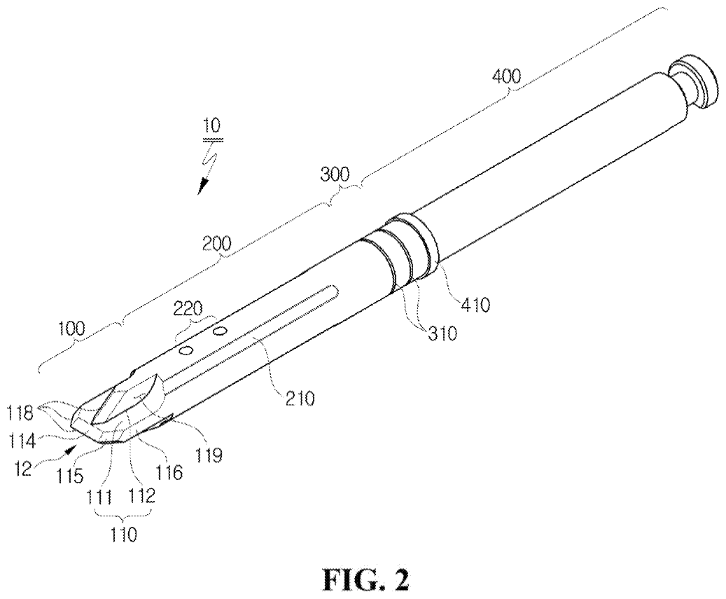

FIG. 2 shows a second embodiment of the present invention. A second inclined portion cutting edge (118) is formed on a second inclined portion (115) of the second cutting element (112) as well.

In the second embodiment shown in FIG. 2, the second inclined portion (115) of the second cutting element (112) serves for an auxiliary cutting action by finishing a hole which is already drilled by the first inclined portion (115) of the first cutting element (111). While the length of the second flat tip (114) of the second cutting element (112) is shorter than the first flat tip (114) of the first cutting element (111), the length of the second inclined portion (115) of the second cutting element (112) is longer than the first inclined portion (115) of the first cutting element (111).

The implant surgical drill bit (10) shown in FIG. 1 or FIG. 2 is suitable in use for an initial drilling into the alveolar bone.

As the first flat tip cutting edge (118) formed on the first flat tip (114) of the first cutting element (111) drills into the bone cortex, the first inclined portion cutting edge (118) formed on the first inclined portion (115) cuts the periphery of the hole to expand the hole size up to the diameter of the implant surgery drill bit (10).

Once a thin bone cortex is drilled out, the inside can be easily drilled through without the need to apply a significant amount of force since the inside is composed of relatively soft cancellous bone.

In FIG. 2, a second inclined portion cutting edge (118) is formed on the second inclined portion (115) of the second cutting element (112). The second inclined portion cutting edge (118) proceeds into the hole which is already made by the first inclined portion (115) of the first cutting element (111) and wraps up the cutting process. Since the first flat tip (114) of the first cutting element (111) is not sharp, the font end (12) of the implant surgery drill bit (10) does not invade the neural tube or the sidewall of the maxillary sinus as even when the front end reaches to a target depth.

On the other hand, the guide part (200) is formed extending from the cutting part (100) and is smoothly connected to the corners (116) of the cutting elements (110). The guide part (200) is in a cylindrical shape and has a constant diameter.

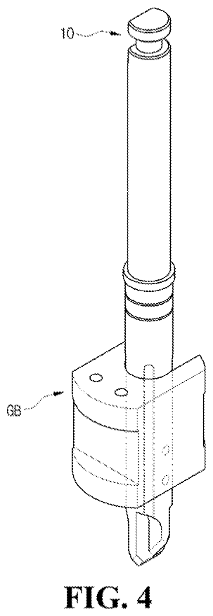

The function of the guide part (200) will be described with reference to the drawings. FIG. 4 shows an implant surgery drill bit coupled with a guide bushing (GB).

The guide bushing (GB) is a hollow cylindrical element and is used in a guided precision surgery. The guide bushing (GB) ensures that the implant surgery drill bit (10) enters in a given position, in a given direction (angle), and to a given depth as pre-planned.

The surgery plan may be established in advance using a computer program and based on the CT image of an oral structure.

Although omitted in FIG. 4, it is embedded in a guide template. The guide template is shaped corresponding to a patient's oral structure and serves as a mouthpiece.

The position and angle of the guide bushing (GB) is fixed in the guide template according to the procedure plan established in the computer program. The guide template is inserted into a patient's oral cavity, and the alveolar bone is drilled by the implant surgery drill bit (10) coupled in the guide bushing (GB). Under this structure, the drilling can be performed precisely according to the pre-established plan. This is the essence of the precise guided implant surgery method.

After all, whether the perforation or drilling of alveolar bone can be performed accurately as planned depends on how accurately the guide bushing (GB) can guide the implant surgical drill bit (10).

Here, the shape of the implant surgery drill bit (10) has a great impact. For example, a tapered drill bit has low guidance precision because it is difficult to accurately contact the inner circumferential surface of the guide bushing (GB).

The implant surgery drill bit (10) of the present invention can enhance the guidance precision in that the guide part (200) is designed in a cylindrical shape with a constant diameter;

Specifically, in a conventional twist drill bit, a spiral flute is provided on the guide part. In contrast, in the present invention, a spiral flute is not provided on the guide part (200) and thus, the guide part can contact the guide bushing (GB) very well.

The structure of the above-described cutting part (100) makes possible this feature, i.e., the spiral flute is not formed on the guide part (200).

Since each cutting element (110) is shaped in a plate with a predetermined thickness, a flute (119) with a considerable size can be provided between two neighboring cutting elements (110). The flute (119) can sufficiently accommodate the bone fragment. Thus, in the present invention, a conventional spiral flute, which is provided on the guide part (200), is no longer necessary.

In the present invention, since no conventional spiral flute is formed on the guide part (200), the guidance precision can enhance; the lateral cutting force of the implant surgery drill bit can be suppressed (10) so that unnecessary wear between the inner surface of the guide bushing (GB) and the implant surgery drill bit (10) can be prevented; the expansion caused by a gap between the drill guide part and the inner surface of the guide busing (GB) can be suppressed.

In an embodiment, the guide part (200) may include some additional components to serve a useful role so long as they do not significantly interfere with the guidance function.

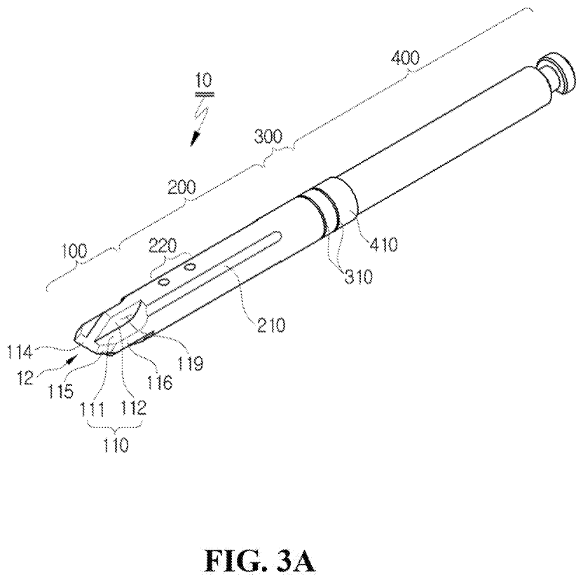

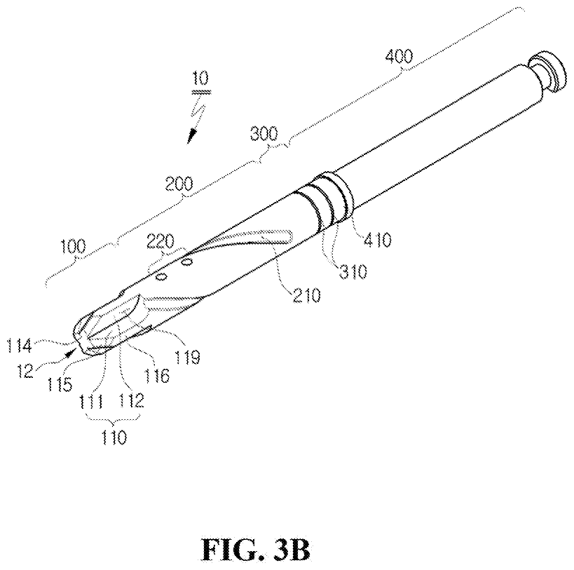

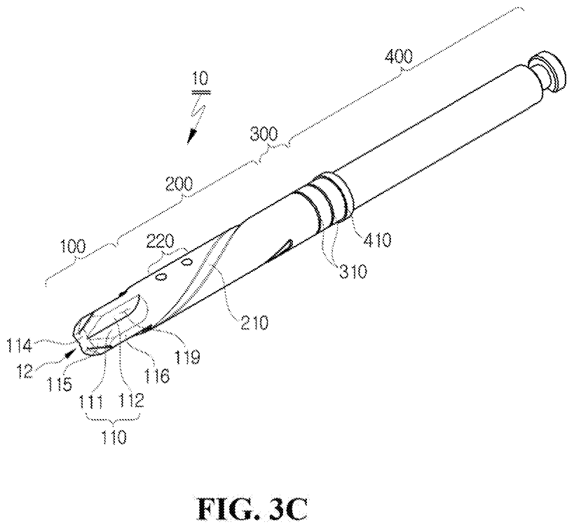

FIG. 3 shows various functional grooves (210) which are formed on the guide part (200).

At least one functional groove (210) is formed as a concave groove along the outer surface of the guide part (200). Multiple functional grooves (210) may be formed to be symmetrical to each other on the guide part (200).

Furthermore, the functional groove (210) may be connected to the flute (119) which is provided between two neighboring cutting elements (110).

For example, each flute (119) may have one corresponding functional groove (210) formed symmetrically.

The boundary between the functional groove (210) and the surface of the guide part (200) may be chamfered or filleted to remove sharp edges.

It is preferable that the functional grooves (210) be arranged symmetrically to each other. The symmetric arrangement of the functional grooves (210) minimizes movement and frictional force that may be caused by the gap between the inner surface of the guide bushing and the drill guide part.

In addition, the functional groove (210) also serves to secure a watering path. Water is supplied to the cutting part (100) through the inside of the flute (119). The water supplied through the functional groove (210) cools the frictional heat generated during the drilling operation; drains blood; and cleans the tissue.

Here, the functional groove (210) formed in the guide part (200) of the present invention should be clearly distinguished from the spiral flute of a conventional twist drill bit.

The functional groove (210) is formed straight along the longitudinal direction of the implant surgery drill bit (10), as shown in FIG. 3A, to facilitate the supply of water. Alternatively, the functional groove (210) may be in a smooth curved shape that forms, for example, a spiral of less than one complete turn, e.g., about ¾ turn as shown in FIGS. 3B and 3C.

The functional groove (210) can be made into a shallow groove in a straight line. This fact shows that the functional groove (210) is not intended to serve to discharge bone fragments from the cutting part (100). Rather, the functional groove (210) serves as a structure that supplies water to the cutting part (100). This makes the functional groove (210) of the present invention distinguished from the spiral flute of a conventional twist drill bit.

In addition, the functional groove (210) is shallow and narrow so that the area occupied in the entire guide part (200) is very small. This structural condition is required to prevent that the functional groove (210) interferes with the guiding function of the guide part (200).

In addition, the guide part (200) may have an engraving (220). The engraving (220) may be of one or more symbols. The engraving may be formed in an intaglio on an outer surface of the guide part. The number of symbols indicates a relative length of a given implant surgery drill bit (10).

When perforating the alveolar bone, the hole does not achieve its final size from the beginning. Rather, a small hole is drilled initially and is gradually expanded over multiple steps until arriving at a final diameter.

To this end, several implant surgery drill bits (10) each having different lengths from each other collectively consists of one set.

However, from a user's perspective, knowing that a given individual drill is supposed to be used at what step of the multi-step surgery is more important than knowing what the exact length is of the given individual drill. In fact, it is more important knowing a relative order in size of a given individual drill than knowing an absolute length of the given individual drill.

The present invention allows the user to quickly and easily recognize the length (more specifically, the relative length of the drill) from the symbol displayed on the surface of the guide part (200). The symbol indicates the length of the implant surgery drill bit (10).

For example, in FIG. 1, two circular figures (spherical shape) are displayed on the guide part (200) of the implant surgery drill bit (10). From this symbol, the user can intuitively recognize that the implant surgery drill bit (10) must be used at a second surgery step. In another embodiment, when a first drill bit which is supposed to be used at a first step is marked with no symbol, a given drill bit marked with two symbols may be a third drill bit which is supposed to be used at a third step. It is preferable that the engraving (220) in a figure shape be formed by a cutting process.

The implant surgery drill bit (10) is fabricated by a cutting process. Thus, the engraving (220) by way of cutting process may be performed simultaneously while the implant surgery drill bit (10) is fabricated. This forgoes an additional process such as laser engraving, printing, or sticker attachment. As a result, production cost can be reduced. The risk of erroneous marking that can result from a separate post-processing can be significantly reduced, as well.

The engraving (220) in a figure shape is made at an appropriate position on the surface of the guide part (200). When the functional groove (210) is formed on the guide part (200), the engraving (220) may be formed in an area where the functional groove (210) is not formed.

Since the functional groove (210) of the present invention is in a straight or has a curved shape that forms a spiral, there is a sufficient empty area between the functional grooves (210) to form the engraving (220).

The peripheral part (300) is connected to the guide part (200) and is not well distinguished from the guide part (200) in shape, as the peripheral part (300) has the same diameter as the guide part (200). However, in consideration of (i) the anatomical structure of a mouth and (ii) the fact that the implant surgery drill bit (10) of the present invention is in use for a precision guided surgery, the peripheral part (300) is necessary.

Since the guide bushing (GB) is used in the precision guided surgery method, an extra length is required in the implant surgery drill bit (10) considering (i) the height of the guide bushing (GB) and (ii) the thickness of the gum covering over the alveolar bone

The height (or length) of the peripheral part (300) is determined in consideration of (i) the height (or length) of the guide bushing (GB) and (ii) the gum thickness of a patient. Since the height of the guide bushing (GB) remains constant, and an anatomical average of the gum thickness is also constant, the height of the peripheral part (300) may be set to a constant value, independent of the height (or length) of individual implant surgery drill bits (10).

The shank part (400) connected to the peripheral part (300) is a shaft fixed to a drill chuck, and has a diameter less than that of the peripheral part (300), so that a step is formed at the boundary between the shank part (400) and the peripheral part (300).

Since the shank part (400) is a general configuration of the implant surgery drill bit (10), a detailed description will be omitted.

In addition, one or more one identification groove (310) may be formed in an intaglio on the circumferential surface of the peripheral part (300). More specifically, the identification groove (310) may be spaced apart by a predetermined distance from the step between the peripheral part (300) and the shank part (400).

The identification groove (310) may be a ring-shaped groove. Similar to the engraving (220) on the guide part (200) described above, the number of identification groove (310) indicates the relative diameter of a given implant surgery drill bit (10).

That is, from the number of the identification grooves (310), the user can immediately understand the relative diameter of the implant surgery drill bit (10). It may be preferable that the identification groove (310) is formed by cutting as well, so that the entire implant surgery drill bit (10) can be formed in one step.

Here, the identification groove (310) is provided at a specific location which is spaced apart by a predetermined distance from the step between the peripheral part (300) and the shank part (400). When there are multiple identification grooves (310), the identification grooves (310) may be arranged spaced from each other at a predetermined interval. With this structure, the identification groove (310) can serve as a scale mark.

For example, assume that the top identification groove (310) is 2 mm away from the step and the interval between the identification grooves (310) is also 2 mm. From the distance between the identification groove and the top surface of the guide bushing (GB), the user can quite accurately estimate how far to go to a target depth.

A user can determine that the target depth is reached and thus conclude perforation, when the step between the peripheral part (300) and the shank part (400) is inserted into the guide bushing (GB). A stopper (410) that extends outward along the step between the peripheral part (300) and the shank part (400) can further aid this determination by restraining the drill bit from further proceeding to drill.

The stopper (410) can reliably prevent the front end (12) of the implant surgery drill bit (10) from invading the neural tube or the sidewall of the maxillary sinus due to excessive perforation.

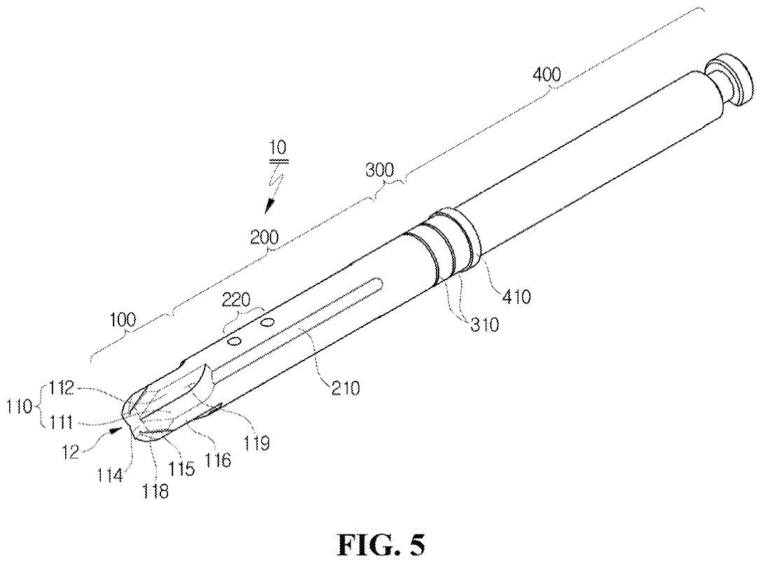

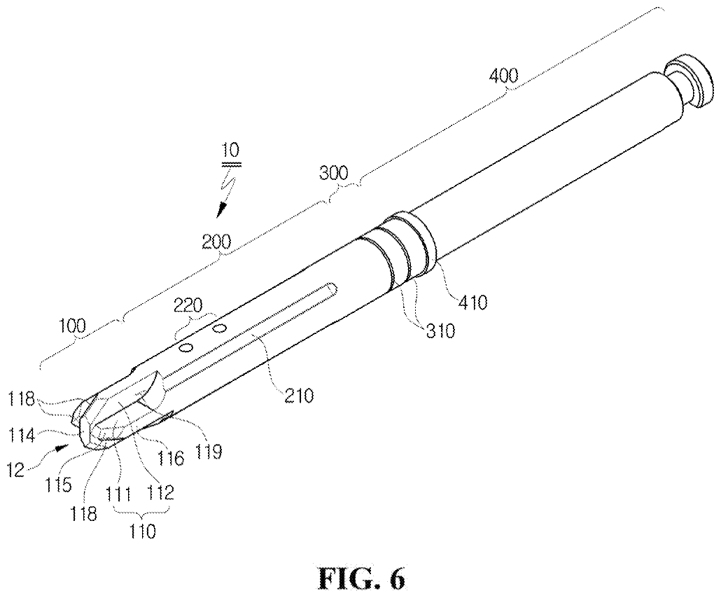

FIGS. 5 to 7 are views showing third to fifth embodiments of the implant surgery drill bit (10) of the present invention.

Here, the third to fifth embodiments are different from the first embodiment and the second embodiment in the configuration of the cutting part (100). Other parts of the embodiments, such as guide parts (200)(including functional grooves and engravings), and the peripheral part (300) (including identification grooves), and the shank part (400) (including the stopper), may be the same as the first and the second embodiments. Therefore, description hereinafter will focus on the cutting part (100) of the third to fifth embodiments.

The third to fifth embodiments is the same as the first embodiment in that (i) each cutting element (110) of the cutting part (100) of the implant surgery drill bit (10) for implant surgery includes the flat tip (114), the inclined portion (115), and the corner (116), (ii) the flute (119) is formed between two neighboring cutting elements (110), and (iii) the cutting edge (118) is arranged at an equal angle along a circumference.

However, there are two important differences. First, the first and the second cutting elements (110) are arranged such that the first flat tip (114) and the second flat tip (114) overlap each other. Under this structure, there is no difference between the height of the first cutting elements (110) and the height of the second cutting elements (110). Second, the first and the second inclined portion cutting edges (118) are formed on the first and the second inclined portions (115) of the first and the second cutting elements (110), respectively.

In addition, in the implant surgery drill bit (10) for implant surgery according to the third to fifth embodiments, there is no need to form a cutting edge (118) on the flat tip (114). This is because the third to fifth embodiments are designed in use for expanding a pre-existing hole that has already formed by drilling.

That is, the implant surgery drill bit (10) of the third to fifth embodiments is designed to expand the pre-existing hole. As the flat tip (114) proceeds into the hole, that has already been drilled, the second inclined portion cutting edge (118) of the second inclined portion (115), which is disposed radially near the flat tip (114), performs a cutting action to expand the hole.

Of course, when the bone cortex of the alveolar bone is considerably thin or weak, the implant surgery drill bit (10) for implant surgery according to the third to fifth embodiments may be used for an initial drilling purpose. However, generally, the drill bit shown the third to fifth embodiments are more suitable for expansion purposes.

In the embodiments shown in FIGS. 5 to 7, the cutting element (110) includes the first cutting element (111) and the second cutting element (112). A first inclined portion cutting edge (118) is formed on a first inclined portion (115) of the first cutting element (111). Likewise, a second inclined portion cutting edge (118) is formed on a second inclined portion (115) of the second cutting element (111).

In addition, in the implant surgery drill bit (10) according to the third to fifth embodiments, the first and the second flat tips (114) of the first and the second cutting elements (110) overlap each other to form a given plane. A center region is provided extending from the center of the flat tip (114) to the end of the shank part (400). The width of the center region is equal to the thickness of each of the cutting elements (110).

Therefore, instead of forming the functional groove (210) on the guide part (200), a water hole (212) can be provided which continuously penetrates from the end of the shank part (400) to the flat tip (114). When the functional groove (210) is not formed on the water hole (212) and replaced with the water hole (212), the guiding function of the guide part (200) improves.

In addition, in FIG. 3, the first cutting edge (118) is formed on the first flat tip (114) of the first cutting element (11l). The first flat tip cutting edge (118) provided on the first flat tip (114) is used to expand a pre-existing hole.

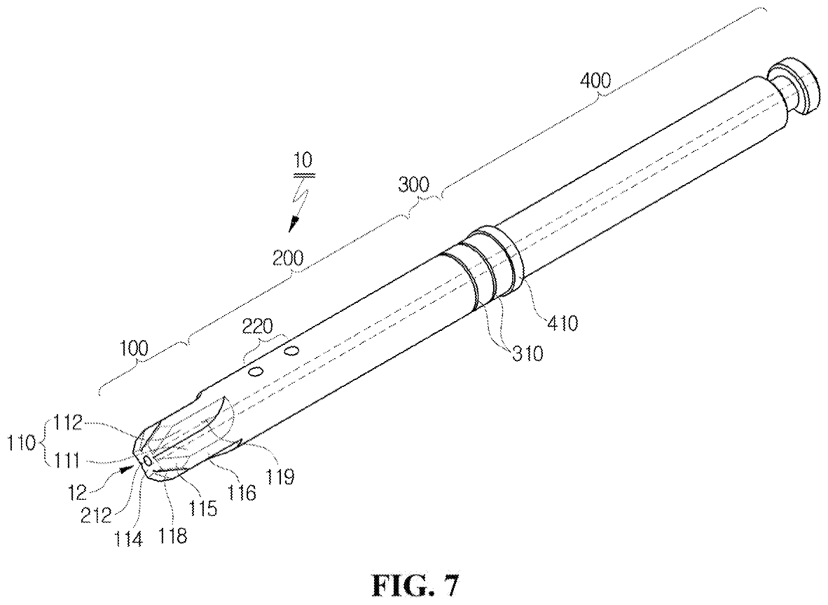

In FIG. 7, the first flat tip cutting edge (118), which is provided on the first flat tip (114) of the first cutting element (111), may be divided into two by the second flat tip (114) of the second cutting element (112). Since the first flat tip cutting edge (118) of the first flat tip (114) serves to cut the periphery of a pre-existing hole, the first flat tip cutting edge (118) does not need to extend to the center of the flat tip (118).

In this case, it is preferable that the first flat tip cutting edge (118) formed on the first flat tip (114) of the first cutting element (111) does not extend beyond the second flat tip (114) of the second cutting element (112). This is advantageous in terms of safety, in maintaining the strength of the divided cutting edges (118) formed on the first flat tip (114), and in protecting the divided cutting edges (118) formed on the first flat tip (114).

The above description is only illustrative of the present invention, and those of ordinary skill in the art to which the present invention pertains will be able to make various modifications and variations without departing from the scope and the spirit of the present invention.

Accordingly, the embodiments of the present invention are provided for explanatory purpose, and are not intended to limit the scope of the present invention. As such, the scope of the present invention should not be limited by these embodiments.

### FIELD OF APPLICATION

The present invention is related to a drill bit suitable in use for drilling a hole in a patient's alveolar bone for dental implant surgery.

What is claimed is:

1. A drill bit for implant surgery comprising:
a cutting part including first and second cutting elements, wherein each of the first and the second cutting elements has a predetermined thickness, wherein a flute is formed between the first and the second cutting elements;
a guide part extending from the cutting part, being in a cylindrical shape, and having a constant diameter;
a peripheral part extending from the guide part, having a predetermined length, and having the same diameter as that of the guide part; and
a shank part extending from the peripheral part and having a different diameter from that of the peripheral part to form a step between the peripheral part and the shank part,
wherein the first cutting element includes a first flat tip, a first inclined portion, and a first corner,
wherein the second cutting element includes a second flat tip, a second inclined portion, and a second corner,
wherein a vertical direction is defined as a longitudinal direction of the drill bit,
wherein a horizontal direction is perpendicular to the vertical direction,
wherein each of the first and the second flat tips extends in a horizontal direction,
wherein the first and the second flat tips overlap each other,
wherein the first and the second inclined portions obliquely extend outward from the first and the second flat tips, respectively,
wherein the first and the second corners extend from the first and the second inclined portions, respectively, along the vertical direction,
wherein each of the first and the second corners has the same diameter as that of the guide part,
wherein no cutting edge is provided on either of the first and the second corners.

2. The drill bit for implant surgery of claim 1,
wherein at least one of the first and the second elements includes a flat tip cutting edge and an inclined portion cutting edge,
wherein the flat tip cutting edge is provided on the first flat tip or on the second flat tip,
wherein the inclined portion cutting edge is provided on the first inclined portion or on the second inclined portion.

3. The drill bit for implant surgery of claim 1,
wherein a first flat tip cutting edge and a first inclined portion cutting edge are provided on the first flat tip and the first inclined portion of the first cutting element, respectively,
wherein the first flat tip cutting edge protrudes the farthest from a front end of the drill bit.

4. The drill bit for implant surgery of claim 3,
wherein a length of the second flat tip of the second cutting element is substantially the same as a thickness of the first flat tip of the first cutting element,
wherein the second flat tip of the second cutting element is buried under the first flat tip cutting edge.

5. The drill bit for implant surgery of claim 4,
wherein a second inclined portion cutting edge is provided on the second inclined portion of the second cutting element.

6. The drill bit for implant surgery of claim 1,
wherein the first flat tip and the second flat tip overlap each other,
wherein a first inclined portion cutting edge is provided on the first inclined portion of the first cutting element,
wherein a second inclined portion cutting edge is provided on the second inclined portion of the second cutting element.

7. The drill bit for implant surgery of claim 6,
wherein a first flat tip cutting edge is formed on the first flat tip of the first cutting element,
wherein the first flat tip cutting edge is divided into two by the second flat tip of the second cutting element.

8. The drill bit for implant surgery of claim 7,
wherein an end of the first flat tip cutting edge of the first cutting element extends up to or shorter than a point where the second flat tip of the second cutting element is located.

9. The drill bit for implant surgery of claim 1, further comprising:
a functional groove concavely formed on an outer surface of the guide part.

10. The drill bit for implant surgery of claim 9,
wherein the functional groove is connected to the flute.

11. The drill bit for implant surgery of claim 10,
wherein the functional groove is (i) in a straight line shape extending along the longitudinal direction of the drill bit, or (ii) in a spiral shape extending around the guide part in a clockwise or counterclockwise direction.

12. The drill bit for implant surgery of claim 11,
wherein the functional groove in the spiral shape is formed shorter than one rotation around the guide part.

13. The drill bit for implant surgery of claim 1, further comprising:
an engraving formed in an intaglio on an outer surface of the guide part,
wherein the engraving includes one or more symbols,
wherein the number of symbols indicates a length of the drill bit.

14. The drill bit for implant surgery of claim 13,
wherein the engraving is formed by a cutting process in which the cutting part is formed.

15. The drill bit for implant surgery of claim 1, further comprising:
an identification groove formed in an intaglio on a circumferential surface of the peripheral part,
wherein the identification groove is spaced apart by a predetermined distance from the step,
wherein the identification groove indicates a diameter of the drill bit.

16. The drill bit for implant surgery of claim 15,
wherein the identification groove is formed by a cutting process in which the cutting part is formed.

17. The drill bit for implant surgery of claim 16,
wherein the identification groove includes multiple grooves,
wherein the multiple grooves are spaced apart from each other by a predetermined interval.

18. The drill bit for implant surgery of claim 1, further comprising:
a stopper protruding outwardly from the step.

19. The drill bit for implant surgery of claim 6,
wherein each of the first flat tip and the second flat tip is a flat surface,
wherein a water hole is provided penetrating from an end of the shank part to the flat surface.

20. The drill bit for implant surgery of claim 1,
wherein no flute is provided on the guide part.
